(19) United States
(12) Patent Application Publication
(10) Pub. No.: US 2026/0188043 A1
(43) Pub. Date: Jul. 2, 2026

(54) Systems and Methods for Identifying Radiation Events in Radiographs

(71) Applicant: IDEXX Laboratories, Inc., Westbrook, ME (US)

(72) Inventors: Benjamin Schestopol, Scarborough, ME (US); Pavan Durgempudi, Portland, ME (US); Todd Joyce, Falmouth, ME (US); Stephanie Mills, Saco, ME (US)

(73) Assignee: IDEXX Laboratories, Inc., Westbrook, ME (US)

(21) Appl. No.: 19/426,189

(22) Filed: Dec. 19, 2025

Related U.S. Application Data
(60) Provisional application No. 63/740,394, filed on Dec. 31, 2024.

(51) Int. Cl. G06V 40/10; A61B 6/00; A61B 6/10; A61B 6/50; G06V 10/776

(52) U.S. Cl. CPC G06V 40/107; A61B 6/10; A61B 6/508; A61B 6/5205; G06V 10/776; G06V 2201/03

(57) ABSTRACT

The present disclosure is related to methods and systems for identifying a user radiation exposure event by providing at least one radiographic image of a patient; using a machine learning algorithm, via execution by a computing device, to process the radiographic image(s) for identification of non-patient anatomy in the radiographic image(s); associating, by the computing device, a label with the identified non-patient anatomy; and generating a data file including the radiographic image(s) of the patient with the labeled identified non-patient anatomy.

### FIELD OF THE DISCLOSURE

This application claims priority to U.S. Provisional Application No. 63/740,394 filed Dec. 31, 2024 which is incorporated herein by reference in its entirety.

### FIELD OF THE DISCLOSURE

The present disclosure involves methods and systems for identifying a user radiation exposure event by radiographers/technicians, e.g. generating machine learning models and artificially intelligent systems for identifying and reporting a user radiation exposure event in the veterinary space.

### BACKGROUND

Radiography is one of the most commonly used diagnostic tools in medical and veterinary practice. Radiographic imaging provides a large amount of information by noninvasive means. It does not alter the disease process or cause unacceptable discomfort to the human or non-human animal. Although radiography itself is painless, sedation is often desirable to reduce anxiety and stress associated with the procedure, to promote acquisition of good diagnostic studies with minimal repeats, and to control pain associated with manipulation in animals with painful disorders such as fractures and arthritis.

However, technicians are sometimes required to hold, restrain or support patients when capturing radiographic images. This is particularly true with patients that cannot or will not comply with instructions regarding positioning, e.g. veterinary or pediatric patients. This can result in the radiographer, e.g. a human technician in a medical or veterinary setting, being exposed to the radiation, e.g. the x-ray beam. Most commonly a radiographer's gloved or ungloved hand is unintentionally exposed to radiation while capturing a radiographic image.

On average veterinary technicians capture between 34 and 48 radiographic images per week. Telemedicine specialists commonly view and read radiographs submitted by veterinary practices. Such telemedicine specialists are obligated to report human anatomy in animal radiographic images as part of their normal interpretation. By far the most common human anatomy accidently exposed are gloved and ungloved hands.

### SUMMARY

Using machine learning it is possible to automatically detect when radiographer (e.g. medical or veterinary doctor or technician) anatomy (e.g. gloved or ungloved hands) appears in a radiographic image of a patient (e.g. a veterinary patient). This can include both gloved and ungloved hands. This will provide real time feedback to the radiographer/technician, aggregate feedback (e.g. for corporate groups) and automate reporting (e.g. for telemedicine specialists). This will result in improved radiation reporting at the clinic and group level and save time for telemedicine specialists.

In an embodiment, the present disclosure relates to a method of identifying a user radiation exposure event comprising the following steps (i) providing at least one radiographic image of a patient, (ii) using a machine learning algorithm, via execution by a computing device, to process the radiographic image(s) for identification of non-patient anatomy in the radiographic image(s); (iii) associating, by the computing device, a label with the identified non-patient anatomy; and (iv) generating a data file including the radiographic image(s) of the patient with the labeled identified non-patient anatomy.

The data file can additionally include a user radiation exposure event notification report. The user radiation exposure event notification report can be automatically sent to predetermined recipients. The non-patient anatomy can be a gloved or ungloved hand. The patient can be a veterinary patient.

The method can further comprise the following steps: (i) inputting at least one training image of patient radiographs known to contain radiographer anatomy; (ii) identifying, by the machine learning algorithm, the presence of radiographer anatomy of one or more training images; (iii) comparing identified presence of radiographer anatomy to known presence of radiographer anatomy; and (iv) adjusting, based on the comparison, the machine learning algorithm.

In an embodiment, the present disclosure also relates to a non-transitory computer-readable medium storing a set of instructions for identifying a user radiation exposure event, where the set of instructions comprises one or more instructions that, when executed by one or more processors of a device, cause the device to complete the steps of the method described above.

In an embodiment, the present disclosure also relates to a system for identifying a user radiation exposure event comprising one or more processors configured to complete the steps of the method described above.

The features, functions, and advantages that have been discussed can be achieved independently in various examples or may be combined in yet other examples. Further details of the examples can be seen with reference to the following description and drawings.

### BRIEF DESCRIPTION OF THE DRAWINGS

The above, as well as additional features will be better understood through the following illustrative and non-limiting detailed description of example embodiments, with reference to the appended drawings. It is noted that like reference characters in different figures refer to the same objects.

FIG. 1 illustrates a simplified block diagram of an example computing device, according to an example embodiment.

FIG. 2 illustrates an example of a radiographic image of a veterinary patient with a labeled human hand according to an example embodiment.

FIG. 3 illustrates a method, according to an example embodiment.

FIG. 4 illustrates a computing system configured for use with an imaging device and a mobile computing device, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

In this regard, in the descriptions herein, certain specific details are set forth to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some embodiments may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

The term “program” in this disclosure should be interpreted to include one or more programs including as a set of instructions or modules that may be executed by one or more components in a system, such as a controller system or data processing device system, in order to cause the system to perform one or more operations. The set of instructions or modules may be stored by any kind of memory device. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

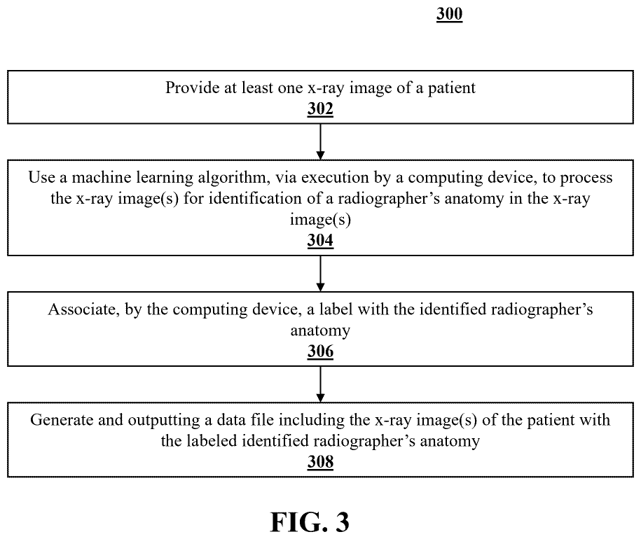

Further still, example methods are described herein with respect to FIG. 3. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangement shown in method 300 herein is not limited to an actual structure of any program or set of instructions or required ordering of method tasks, and such method 300, according to some embodiments, merely illustrates the tasks that instructions are configured to perform, for example upon execution by a data processing device system in conjunction with interactions with one or more other devices or device systems.

The present disclosure involves methods and systems for identifying a user radiation exposure event by radiographers/technicians, e.g. generating machine learning models and artificially intelligent systems for identifying and reporting a user radiation exposure event. Using machine learning it is possible to automatically detect when radiographer (e.g. medical or veterinary doctor or technician) anatomy (e.g. gloved or ungloved hands) appears in a radiographic image of a patient (e.g. a veterinary patient).

Within examples, the present disclosure is directed to a method of identifying a user radiation exposure event comprising the following steps (i) providing at least one radiographic image of a patient, (ii) using a machine learning algorithm, via execution by a computing device, to process the radiographic image(s) for identification of non-patient, e.g. a radiographer's anatomy in the radiographic image(s); (iii) associating, by the computing device, a label with the identified non-patient anatomy; and (iv) generating a data file including the radiographic image(s) of the patient with the labeled identified non-patient anatomy.

The method can further comprise the following steps: (i) inputting at least one training image of patient radiographs known to contain radiographer anatomy; (ii) identifying, by the machine learning algorithm, the presence of radiographer anatomy of one or more training images; (iii) comparing identified presence of radiographer anatomy to known presence of radiographer anatomy; and (iv) adjusting, based on the comparison, the machine learning algorithm.

In example embodiments, the radiographic patient images may include one or more of the following: x-rays or plain radiographs e.g. of bones, the abdomen, the chest or the jaw/teeth, computed axial tomography (CAT or CT scans), magnetic resonance imaging (MRI), ultrasound, fluoroscopy, and molecular imaging among other possibilities. Other examples are possible.

In example embodiments, the patient may include human or non-human animals. The present application is particularly useful with patients that cannot or will not comply with instructions related to positioning within the radiographic device. For example, veterinary patients, e.g. companion (cat, dog, horse, rodent, reptile, etc.) or livestock (cow, pig, chicken, etc.) non-human animals, typically need to be held in certain physical orientations during radiography which increases the likelihood of non-patient, e.g. radiographer/technician/clinician, anatomy appearing in the radiographic image.

In example embodiments, the non-patient anatomy may include a gloved or ungloved human hand. Challenges previously existed with identifying both human gloved and ungloved anatomy. Other examples are possible.

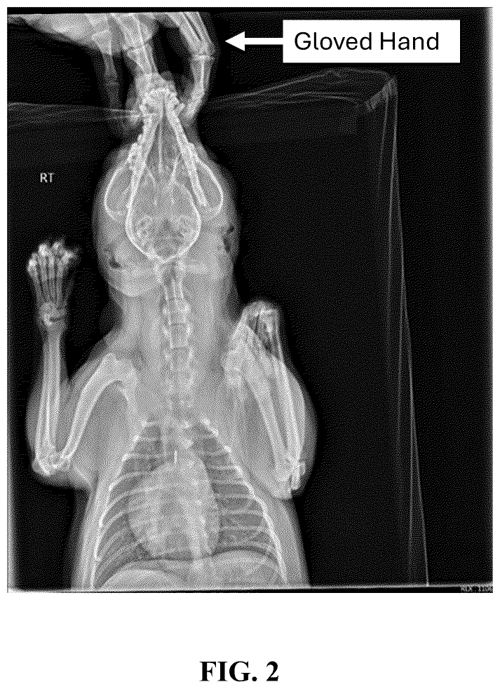

FIG. 2 illustrates an example of a radiographic image of a veterinary patient with a labeled human hand according to an example embodiment. The radiographic image of the veterinary patient appears on the image with the human hand appearing in the upper left corner of the image.

The present method associates, by a computing device, a label with an identified non-patient anatomy. The computing device can then generate and output a data file that includes the radiographic image of the patient with the labeled non-patient anatomy This can provide real time feedback to the radiographer/clinician/technician to allow for remediation.

For example, the system uses a machine learning algorithm to process radiographic images and identify non-patient anatomy, such as a human hand (gloved or ungloved), that appears in the image. Once identified, the system associates a label with the detected hand. This label is then embedded in the image data, marking the location and presence of the hand for further review or reporting.

The label can be as simple as a bounding box or marker over the hand in the radiograph, or it can be a more complex annotation that includes metadata (e.g., “gloved hand detected,” “ungloved hand detected,” “technician's hand”). In practice, the labeled hand might appear as an overlay or highlighted region on the radiograph, with a tag or note indicating “human hand” or “technician's hand.” This visual cue helps radiographers and reviewers quickly identify and address accidental exposures.

The data file can additionally include a user radiation exposure event notification report. The report can contain details regarding the radiation exposure event. For example, it could contain identifying information for the clinic, e.g. name, address, phone number, identification number, e-mail address, etc. The report could also contain similar identifying information for the exposed radiographer, e.g. name, address, phone number, identification number, e-mail address, gender, age, etc. The report could include information related to the exposure, e.g. length of time, type of radiograph, amount of exposure, cumulative amount of exposure for that technician in a predetermined period of time, etc. The user radiation exposure event notification report can be automatically sent to predetermined recipients, e.g. clinic management, corporate management, government agencies for reporting requirements, etc. This can provide real time aggregate feedback (e.g. for corporate groups) and automate reporting (e.g. for telemedicine specialists). This will result in improved radiation reporting at the clinic and group level and save time for telemedicine specialists.

In further embodiments, the disclosed system and methods are configured to measure the area or size of the irradiated human anatomy detected within a radiographic image. For example, the system may utilize image analysis algorithms to quantify the portion of anatomy exposed, such as distinguishing between two fingers, half of a hand, or an entire hand. This measurement may be performed by calculating the pixel area corresponding to the labeled anatomy and correlating it with known anatomical dimensions, thereby providing an estimate of the actual physical area exposed.

In yet another embodiment, the system and method are further configured to estimate the total dosage of irradiation received by the identified anatomy. This estimation may be based on the measured area of exposure, the duration of exposure, and parameters of the irradiation source, such as the energy level, intensity, and exposure time. The system may retrieve or receive irradiation source data from the imaging device or associated metadata, and apply established dosimetric models to calculate an estimated radiation dose for the exposed anatomy. This information may be included in the user radiation exposure event notification report.

Additionally, the system may be configured to aggregate and report radiation exposure events by user or technician. In such embodiments, the system associates each detected exposure event with a specific user or technician, for example, by linking user credentials, device logins, or manual input at the time of image acquisition. The system may maintain a database of exposure events for each user, including details such as date, time, anatomical area exposed, estimated dosage, and the imaging procedure performed. Periodic or on-demand reports may be generated to summarize cumulative exposure for individual users, support compliance with regulatory requirements, and facilitate occupational health monitoring.

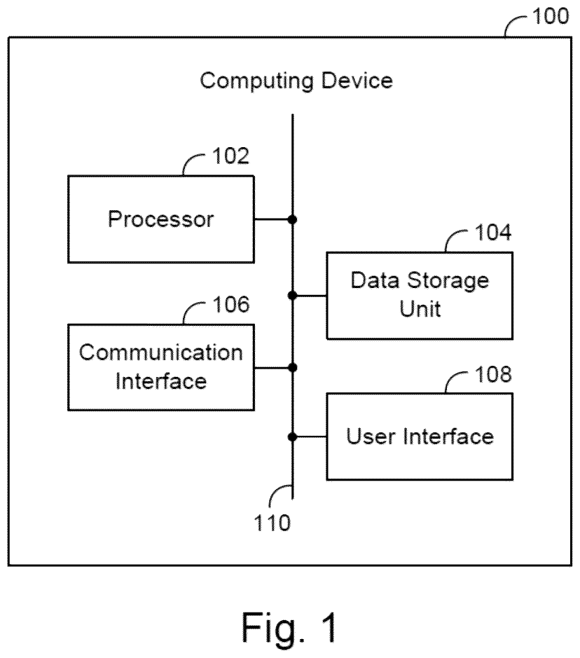

FIG. 1 is a simplified block diagram of an example computing device 100 of a system (e.g., that can be utilized with devices and methods illustrated in FIGS. 2-4, described in further detail below). Computing device 100 can perform various acts and/or functions, such as those described in this disclosure. Computing device 100 can include various components, such as processor 102, data storage unit 104, communication interface 106, and/or user interface 108. These components can be connected to each other (or to another device, system, or other entity) via connection mechanism 110.

Processor 102 can be implemented in hardware, firmware, or a combination of hardware and software. The processor 102 can include one or more processing devices that implement or execute, in conjunction with other devices, such as one or more of those in the computing device 100, control programs associated with some of the various embodiments. Each of the phrases “data processing device”, “data processor”, “processor”, and “computer” is intended to include any processor, such as a central processing unit (“CPU”), a desktop computer, a laptop computer, a mainframe computer, a tablet computer, a personal digital assistant, a cellular phone, and any other device configured to process data, manage data, or handle data, whether implemented with electrical, magnetic, optical, biological components, or other. Processor 102 can include a general-purpose processor (e.g., a microprocessor and/or a central processing unit (CPU)) and/or a special-purpose processor (e.g., a digital signal processor (DSP) and/or a graphics processing unit (GPU)).

Data storage unit 104 can include one or more volatile, non-volatile, removable, and/or non-removable storage components, such as magnetic, optical, or flash storage, and/or can be integrated in whole or in part with processor 102. The data storage unit 104 can include one or more processor-accessible memory devices configured to store information, including the information needed to execute the control programs associated with some of the various embodiments. The data storage unit 104 may be a distributed processor-accessible memory device system including multiple processor-accessible memory devices communicatively connected to the processor 102 via a plurality of computers and/or devices. On the other hand, the data storage unit 104 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some embodiments, each of the phrases “processor-accessible memory” and “processor-accessible memory device” is intended to include a non-transitory computer-readable storage medium.

Further, data storage unit 104 can take the form of a non-transitory computer-readable storage medium, having stored thereon program instructions (e.g., compiled or non-compiled program logic and/or machine code) that, when executed by processor 102, cause computing device 100 to perform one or more acts and/or functions, such as those described in this disclosure. As such, computing device 100 can be configured to perform one or more acts and/or functions, such as those described in this disclosure. Such program instructions can define and/or be part of a discrete software application. In some instances, computing device 100 can execute program instructions in response to receiving an input, such as from communication interface 106 and/or user interface 108. Data storage unit 104 can also store other types of data, such as those types described in this disclosure.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the data storage unit 104 is shown separately from the processor 102 and the communication and user interfaces 106 and 108, one skilled in the art will appreciate that the data storage unit 104 may be located completely or partially within the processor 102 or the communication and user interfaces 106 and 108. Further in this regard, although the communication and user interfaces 106 and 108 are shown separately from the processor 102 and the data storage unit 104, one skilled in the art will appreciate that such system may be located completely or partially within the processor 102 or data storage unit 104, depending upon the contents of the communication and user interfaces 106 and 108. Further still, the processor 102, communication and user interfaces 106 and 108, and the data storage unit 104 may be located entirely within the same device or housing or may be separately located, but communicatively connected, among different devices or housings. In the case where the processor 102, communication and user interfaces 106 and 108, and the data storage unit 104 are located within the same device, the system 100 of FIG. 1 can be implemented by a single application-specific integrated circuit (ASIC) in some embodiments.

Communication interface 106 can allow computing device 100 to connect to and/or communicate with another other entity according to one or more protocols. In one example, communication interface 106 can be a wired interface, such as an Ethernet interface or a high-definition serial-digital-interface (HD-SDI). In another example, communication interface 106 can be a wireless interface, such as a cellular or WI FI interface. In this disclosure, a connection can be a direct connection or an indirect connection, the latter being a connection that passes through and/or traverses one or more entities, such as a router, switcher, or other network device. Likewise, in this disclosure, a transmission can be a direct transmission or an indirect transmission.

User interface 108 can facilitate interaction between computing device 100 and a user of computing device 100, if applicable. As such, user interface 108 can include input components such as a keyboard, a keypad, a mouse, a touch sensitive panel, a microphone, a camera, and/or a sensor (e.g., a GPS component, an accelerometer, a gyroscope, and/or an actuator), all of which can be used to obtain data indicative of an environment of computing device 100, and/or output components such as a display device (which, for example, can be combined with a touch sensitive panel), a sound speaker, and/or a haptic feedback system. More generally, user interface 108 can include hardware and/or software components that facilitate interaction between computing device 100 and the user of the computing device 100.

Computing device 100 may also contain a battery module to supply power to processor 102, data storage unit 104, and internal components of computing device 1009. The battery module may supply power during field measurements by computing device 100. The battery module also permits computing device 100 to be a portable integrated device for conducting field measurements of propagation delay in a RAN.

Computing device 100 can take various forms, such as a workstation terminal, a desktop computer, a laptop, a tablet, a mobile phone, or a controller. Computing device 100 may perform one or more processes described herein. Computing device 100 may perform these processes by processor 102 executing software instructions stored by a non-transitory computer-readable medium, such as data storage unit 104.

Software instructions may be read into data storage unit 104 from another computer-readable medium or from another device a communication interface 106. When executed, software instructions stored in data storage unit 104 may instruct processor 102 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 1 are provided as an example. In practice, computing device 100 may include additional components, fewer components, different components, or differently arranged components than those shown in FIG. 1. Additionally, or alternatively, a set of components (e.g., one or more components) of computing device 100 may perform one or more functions described as being performed by another set of components.

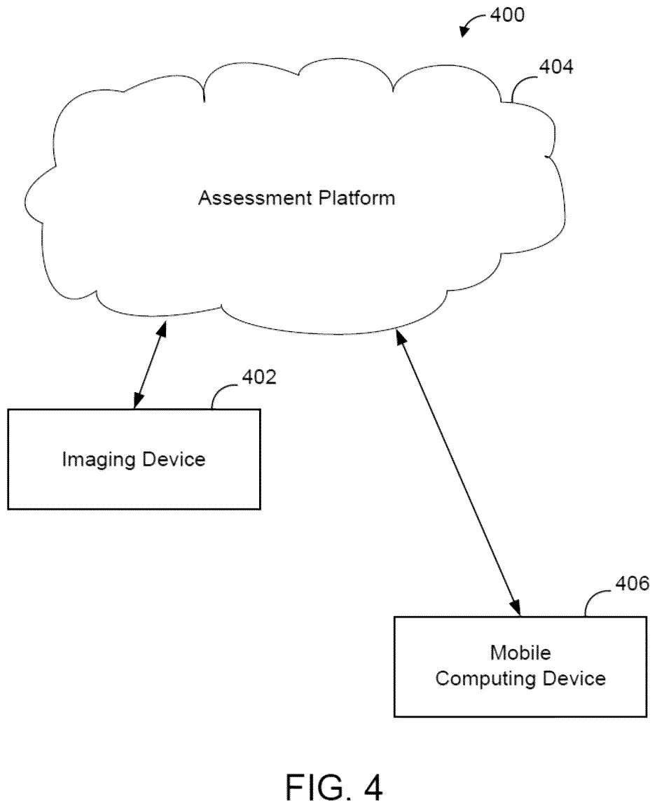

Now referring to FIG. 4, a computing system 400 configured for use with an imaging device 402 and a mobile computing device 406, according to an example embodiment. An imaging device 402 includes a computing device, such as computing device 100. It should also be readily understood that computing device 100 and the imaging device 402, and all of the components thereof, can be physical systems made up of physical devices, cloud-based systems made up of cloud-based devices that store program logic and/or data of cloud-based applications and/or services (e.g., perform at least one function of a software application or an application platform for computing systems and devices detailed herein), or some combination of the two.

A cloud computing system can include computing hardware, a resource management component, a host operating system (OS), and/or one or more virtual computing systems. The resource management component may perform virtualization (e.g., abstraction) of the computing hardware to create the one or more virtual computing systems. Using virtualization, the resource management component enables a single computing device (e.g., a computer, a server, and/or the like) to operate like multiple computing devices, such as by creating multiple isolated virtual computing systems from the computing hardware of the single computing device. In this way, the computing hardware can operate more efficiently, with lower power consumption, higher reliability, higher availability, higher utilization, greater flexibility, and lower cost than using separate computing devices.

The computing hardware can include hardware and corresponding resources from one or more computing devices. For example, the computing hardware may include hardware from a single computing device (e.g., a single server) or from multiple computing devices (e.g., multiple servers), such as multiple computing devices in one or more data centers. The computing hardware may include one or more processors, one or more memories, one or more storage components, and/or one or more networking components.

The resource management component includes a virtualization application (e.g., executing on hardware, such as the computing hardware) capable of virtualizing the computing hardware to start, stop, and/or manage the one or more virtual computing systems. For example, the resource management component may include a hypervisor (e.g., a bare-metal or Type 1 hypervisor, a hosted or Type 2 hypervisor, and/or the like) or a virtual machine monitor, such as when the virtual computing systems are virtual machines. Additionally, or alternatively, the resource management component may include a container manager, such as when the virtual computing systems are containers. In some implementations, the resource management component executes within and/or in coordination with a host operating system.

A virtual computing system includes a virtual environment that enables cloud-based execution of operations and/or processes described herein using computing hardware. As shown, the virtual computing system may include a virtual machine, a container, a hybrid environment that includes a virtual machine and a container, and/or the like. A virtual computing system may execute one or more applications using a file system that includes binary files, software libraries, and/or other resources required to execute applications on a guest operating system (e.g., within the virtual computing system) or the host operating system.

The network includes one or more wired and/or wireless networks. For example, the network may include a cellular network, a public land mobile network (PLMN), a local area network (LAN), a wide area network (WAN), a private network, the Internet, and/or the like, and/or a combination of these or other types of networks. The network enables communication among the devices of the environment.

The base station may support, for example, a cellular radio access technology (RAT). The base station may include one or more base stations (e.g., base transceiver stations, radio base stations, node Bs, eNodeBs (eNBs), gNodeBs (gNBs), base station subsystems, cellular sites, cellular towers, access points, transmit receive points (TRPs), radio access nodes, macrocell base stations, microcell base stations, picocell base stations, femtocell base stations, or similar types of devices) and other network entities that can support wireless communication for the user device. The base station may transfer traffic between the user device (e.g., using a cellular RAT), one or more base stations (e.g., using a wireless interface or a backhaul interface, such as a wired backhaul interface), and/or a core network. The base station may provide one or more cells that cover geographic areas.

The user device includes one or more devices capable of receiving, generating, storing, processing, and/or providing information, as described elsewhere herein. The user device may include a communication device and/or a computing device. For example, the user device may include a wireless communication device, a mobile phone, a user equipment, a laptop computer, a tablet computer, a desktop computer, a gaming console, a set-top box, a wearable communication device (e.g., a smart wristwatch, a pair of smart eyeglasses, a head mounted display, or a virtual reality headset), or a similar type of device.

In any event, a computing system 400 can include various components, such as the computing device 100, imaging device 402, a cloud-based assessment platform 404.

The imaging device 402 and/or components thereof can perform various acts and/or functions (many of which are described above). Examples of these and related features will now be described in further detail.

The imaging device 402 may collect data from a number of sources. In one example, the imaging device 402 may collect data from a database of images related to patient radiographic images, including one or more images of patient radiographic images with and without radiographer anatomy in the radiographic images. The images may be uploaded to an assessment platform 404 and identification of the presence of non-patient anatomy may be output to a mobile computing device 406.

In an example, assessment platform 404 may collect data from one or more sensors communicably coupled to the imaging device 402, such as an imaging sensor, concerning a particular patient radiographic image. In such examples, the assessment platform 404 may identify the presence of a non-patient anatomy in the patient radiographic image and transmit instructions to the mobile computing device 406 to cause a graphical user interface to display a graphical indication of the presence of a non-patient anatomy. In some examples, the assessment platform 404 may determine the presence of a non-patient anatomy in the patient radiographic image by utilizing one or more of: (i) an artificial neural network, (ii) a support vector machine, (iii) a regression tree, or (iv) an ensemble of regression trees.

In another example, the imaging device 402 may collect data from one or more sensors communicably coupled to the imaging device, such as an imaging sensor, concerning a particular patient radiographic image. In some examples, the assessment platform 404 may determine the presence of non-patient anatomy in the patient radiographic image by utilizing one or more of: (i) an artificial neural network, (ii) a support vector machine, (iii) a regression tree, or (iv) an ensemble of regression trees.

In some examples, images that are captured by the imaging device can be stored within a memory, such as a memory of computing device 100, to be subsequently analyzed.

In another example, the imaging device 402 may collect data from a plurality of sensors of the imaging device and superimpose the data. For example, assessment platform 404 may collect data in the form of multiple monochromatic images (e.g., from different wavelength sources) from an imaging sensor of imaging device 402 and then overlay the monochromatic images. In some examples, assessment platform 404 may collect data from a sensor of the imaging device 402 and input data from a user of the mobile computing device 406 or a user of the imaging device 402. In one example, assessment platform 404 may transmit instructions to cause a graphical user interface to display a graphical indication of the presence of non-patient anatomy in the patient radiographic image along with the input data received from a user of the mobile computing device 406 or a user of the imaging device 402.

In one example, the imaging device 402 may train a machine learning model using data associated with images of patient radiographs that share a characteristic, e.g. the presence of radiographer anatomy, with captured images of patient radiographs. The machine learning model may be trained using training data that shares a characteristic, e.g. the presence of radiographer anatomy, with a patient radiograph to be analyzed by the imaging device. Training the machine learning model may include inputting one or more training images into the machine learning model, predicting, by the machine learning model, an outcome of a determined condition of the one or more training images, comparing the at least one outcome to the characteristic of the one or more training images, and adjusting, based on the comparison, the machine learning model. For example, if a user is attempting to identify radiographer anatomy in patient radiographs, the machine learning model may be trained by inputting images of patient radiographs known to contain radiographer anatomy (e.g. gloved and ungloved hands), identifying, by the machine learning model, the presence of radiographer anatomy (e.g. gloved and ungloved hands) of one or more training images, comparing the identified presence of radiographer anatomy (e.g. gloved and ungloved hands) to the known presence of radiographer anatomy (e.g. gloved and ungloved hands), and adjusting, based on the comparison, the machine learning model.

In some examples, the training data may include labeled input images (supervised learning), partially labeled input images (semi-supervised learning), or unlabeled input images (unsupervised learning). In some examples, training may include reinforcement learning.

The machine learning model may include an artificial neural network, a support vector machine, a regression tree, an ensemble of regression trees, or some other machine learning model architecture or combination of architectures.

The training data may include images of patient radiographs with and without radiographer anatomy (e.g. gloved and ungloved hands), data associated with said patient radiographs, blank images, synthetic images, augmented images, or any combination thereof.

In some examples, the machine learning model of the imaging device 402 may be adjusted based on training such that if the outcome of a determined condition matches the characteristic (e.g. the presence of a radiographer's anatomy) of the training images, the machine learning model is reinforced and if the outcome of a determined condition does not match the characteristic (e.g. the presence of a radiographer's anatomy) of the training images, the machine learning model is modified. In some examples, modifying the machine learning model includes increasing or decreasing a weight of a factor within the neural network of the machine learning model. In other examples, modifying the machine learning model includes adding or subtracting rules during the training of the machine learning model.

Once the imaging device 402 has determined the presence of non-patient anatomy in the patient radiographic image in one or more images, the imaging device may transmit instructions that cause a computing device (e.g., the computing device 100) to display one or more graphical indications of the identified characteristic and/or the enhanced image.

In an example, an imaging device (such as imaging device 402 shown in FIG. 4) can train a machine learning model using data associated with images of the same or similar composite targets. Generally, training the machine learning model may include inputting one or more training images of the composite targets into the machine learning model, predicting, by the machine learning model, an outcome of a determined condition of the one or more training images, comparing the at least one outcome to the characteristic of the one or more training images, and adjusting, based on the comparison, the machine learning model. Additionally or alternatively, in some examples, computer vision techniques can also be employed to identify and process characteristics of a target in an image.

In example implementations, once parameters of the imaging device are determined and assessed, these parameters can be used to calibrate the imaging device. The imaging device can store these parameters (e.g., via a memory of computing device 100) to be utilized and/or applied during subsequent imaging.

### EXAMPLE METHODS AND ASPECTS

Now referring to FIG. 3, an example method of identifying a user radiation exposure event. Method 300 shown in FIG. 3 presents an example of a method of identifying a user radiation exposure event that could be used such as the example systems and/or imaging device shown in FIG. 1 or 4, for example. Further, devices or systems may be used or configured to perform logical functions presented in FIG. 3.

FIG. 3 is a flowchart of an example process 300. In some implementations, one or more process blocks of FIG. 3 may be performed by a device.

As shown in FIG. 3, process 300 may include providing at least one radiographic image of a patient (block 302). For example, device may provide at least one radiographic image of a patient, as described above. As also shown in FIG. 3, process 300 may include using a machine learning algorithm, via execution by a computing device, to process the radiographic image(s) for identification of a radiographer's anatomy in the radiographic image(s) (block 304). For example, device may use a machine learning algorithm, via execution by a computing device, to process the radiographic image(s) for identification of a radiographer's anatomy in the radiographic image(s), as described above. As further shown in FIG. 3, process 300 may include associating, by the computing device, a label with the identified radiographer's anatomy (block 306). For example, device may associate, by the computing device, a label with the identified radiographer's anatomy, as described above. As also shown in FIG. 3, process 300 may include generating and potentially outputting a data file including the radiographic image(s) of the patient with the labeled identified radiographer's anatomy (block 308). For example, the device may generate and potentially output a data file including the radiographic image(s) of the patient with the labeled identified radiographer's anatomy, as described above.

In other examples, components of the devices and/or systems may be arranged to be adapted to, capable of, or suited for performing the functions, such as when operated in a specific manner. Method 300 may include one or more operations, functions, or actions as illustrated by one or more of blocks 302-308. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

Subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

What is claimed is:

1. A method of identifying a user radiation exposure event comprising:
providing at least one radiographic image of a patient;
using a machine learning algorithm, via execution by a computing device, to process the radiographic image(s) for identification of non-patient anatomy in the radiographic image(s);
associating, by the computing device, a label with the identified non-patient anatomy; and
generating a data file including the radiographic image(s) of the patient with the labeled identified non-patient anatomy.

2. The method of claim 1 wherein said data file additionally includes a user radiation exposure event notification report.

3. The method of claim 1 wherein the user radiation exposure event notification report is automatically sent to predetermined recipients.

4. The method of claim 1 wherein the non-patient anatomy is a gloved hand.

5. The method of claim 1 wherein the non-patient anatomy is an ungloved hand.

6. The method of claim 1 wherein the patient is a veterinary patient.

7. The method of claim 1 further comprising
inputting at least one training image of patient radiographs known to contain non-patient anatomy;
identifying, by the machine learning algorithm, the presence of non-patient anatomy of one or more training images;
comparing identified presence of non-patient anatomy to known presence of non-patient anatomy; and
adjusting, based on the comparison, the machine learning algorithm.

8. A non-transitory computer-readable medium storing a set of instructions for identifying a user radiation exposure event, the set of instructions comprising:
one or more instructions that, when executed by one or more processors of a device, cause the device to:
provide at least one radiographic image of a patient;
use a machine learning algorithm, via execution by a computing device, to process the radiographic image(s) for identification of non-patient anatomy in the radiographic image(s);
associate, by the computing device, a label with the identified non-patient anatomy; and
generate a data file including the radiographic image(s) of the patient with the labeled identified non-patient anatomy.

9. The non-transitory computer-readable medium of claim 8, wherein said data file additionally includes a user radiation exposure event notification report.

10. The non-transitory computer-readable medium of claim 8, wherein the user radiation exposure event notification report is automatically sent to predetermined recipients.

11. The non-transitory computer-readable medium of claim 8, wherein the non-patient anatomy is a gloved hand.

12. The non-transitory computer-readable medium of claim 8, wherein the non-patient anatomy is an ungloved hand.

13. The non-transitory computer-readable medium of claim 8, wherein the patient is a veterinary patient.

14. The non-transitory computer-readable of claim 8 wherein the one or more instructions further cause the device to
input at least one training image of patient radiographs known to contain non-patient anatomy;
identify, by the machine learning algorithm, the presence of non-patient anatomy of one or more training images;
compare identified presence of non-patient anatomy to known presence of non-patient anatomy; and
adjust, based on the comparison, the machine learning algorithm.

15. A system for identifying a user radiation exposure event comprising one or more processors configured to:
provide at least one radiographic image of a patient;
use a machine learning algorithm, via execution by a computing device, to process the radiographic image(s) for identification of non-patient anatomy in the radiographic image(s);
associate, by the computing device, a label with the identified non-patient anatomy; and
generate a data file including the radiographic image(s) of the patient with the labeled identified non-patient anatomy.

16. The system of claim 15, wherein said data file additionally includes a user radiation exposure event notification report.

17. The system of claim 15, wherein the user radiation exposure event notification report is automatically sent to predetermined recipients.

18. The system of claim 15, wherein the non-patient anatomy is a gloved hand.

19. The system of claim 15, wherein the non-patient anatomy is an ungloved hand.

20. The system of claim 15, wherein the patient is a veterinary patient.

21. The system of claim 15 wherein the one or more processors are further configured to:
input at least one training image of patient radiographs known to contain non-patient anatomy;
identify, by the machine learning algorithm, the presence of non-patient anatomy of one or more training images;
compare identified presence of non-patient anatomy to known presence of non-patient anatomy; and
adjust, based on the comparison, the machine learning algorithm.
